(19) United States
(12) Patent Application Publication
(10) Pub. No.: US 2009/0000317 A1
(43) Pub. Date: Jan. 1, 2009

(54) Device and method for disinfection of ice machines, ice silos and/or chutes for transport of ice

(76) Inventors: Joachim Schill, Kehl (DE); Joachim Roos, Offenburg (DE)

Correspondence Address: ORUM & ROTH, Ste 1616, 53 West Jackson Boulevard, Chicago, IL, 60604, US

(21) Appl. No.: 12/079,549

(22) Filed: Mar. 27, 2008

(30) Foreign Application Priority Data
Mar. 29, 2007 (DE) 102007015584.2

(51) Int. Cl. F24F 3/16; B05B 9/04; B05B 1/24; B05B 15/00; A61L 2/20

(52) U.S. Cl. 62/78; 239/373; 239/135; 239/338; 422/28

(57) ABSTRACT

Device and method for disinfection of ice machines, ice silos, and/or chutes for transport of the ice, with a storage tank (8) for holding a disinfectant, with a pressure line (7) for the supply of pressurized carrier fluid, with at least one atomizer device (6) for atomizing the disinfectant and for generating a mixture of carrier fluid and disinfectant and with an inlet for introducing the carrier fluid/disinfectant mixture into the ice machine (1, 19, 23, 28, 32), the ice silo (2, 20, 24, 33), or the ice chutes (4, 21, 29, 34).

### PRIOR ART

The invention is based upon a device and a method for the disinfection of ice machines, ice silos, and/or ice chutes for the transport of ice, and of ice machines and ice silos which are equipped with devices for disinfection.

Ice machines are used for making ice from any liquids. In this process, the ice can have different shapes, such as thin sheets, cubes, flakes, liquid ice, or granular particles. Water is frequently used as a liquid for making ice. The ice produced with water is used in the manufacture of foodstuffs and for the preservation of foodstuffs during transportation and storage. In this manner, meat, fish, or seafood can, for example, be stored and transported without a loss of quality. Ice in the form of thin sheets, which may also be referred to as flake ice, is used during the manufacture of sausage. In addition to water, other liquids such as juices, sauces, eggs, milk, and milk products can also be made into ice. The ice manufactured in larger facilities by means of an ice machine is collected in so-called ice silos, before it is moved for further processing or to the point of use. The ice silos are usually equipped with large containers, into which the ice produced by the ice machine is carried by means of channels, also referred to as ice chutes. The ice silos are frequently thermally insulated in order to prevent the ice from melting during storage.

Because the ice is used for the manufacture of foodstuffs, the ice machine for making the ice and the ice silo for collection and storage of the ice are subject to particularly stringent requirements in terms of hygiene. One requirement is that all surfaces coming into contact with the ice must be free of pathogens, in particular bacteria, viruses, fungi and protozoa. For this purpose, the respective surfaces of the ice machine, of the ice silo and the chutes which connect the ice machine with the ice silo, must be disinfected at specific time intervals.

DE 4108911 A1 discloses an ice machine with a rotating freezing cylinder and a tank surrounding the freezing cylinder, provided with a controllable cleaning device for rinsing the tank and the freezing cylinder. The cleaning device has several spray nozzles for spraying the freezing cylinder and the tank with a cleaning agent. In this regard, a disadvantage is that only those parts of the ice machine that are within the spray cone of the spray nozzles are cleaned. In order to appropriately clean the entire freezing cylinder and the tank, a large number of spray nozzles is needed. The device cleans only the parts of the ice machine used for making the flake ice. The devices used for collecting and transporting the flake ice are not cleaned. In addition, a special cleaning fluid is used for cleaning, which is removed from the ice machine through the drain of the tank, and any of its residues have to be eliminated from the ice machine by subsequent rinsing. The cleaning can therefore only be carried out as long as no ice is being produced and as long as no ice and no liquid to be frozen are in the ice machine. Cleaning is not possible while ice is being produced.

DE 19821284 A1 discloses a flake ice machine which is provided with at least one UV light source for cleaning, disinfecting, and sterilizing the flake ice. This UV light source may be located at the freezing cylinder, at the device for separating the flake ice from the lateral surface of the freezing cylinder, at the conveyor device, and at the collection device for collecting the ice that has been produced. Even though the UV light source allows for continuous disinfection of the ice, the UV light source has, however, the disadvantage of being very expensive. The ultraviolet rays are absorbed by the DNA of the bacteria and fungi. Thus, the DNA structure is destroyed and the microorganisms are killed. This process depends on the intensity of the UV irradiation. Consequently, for disinfection of all surfaces of the ice machine, the ice silo, and the chutes with which the ice comes into contact, this requires the direct illumination of these surfaces with a UV light source. Because of its low intensity, the stray light is insufficient for the disinfection. For this reason, a large number of UV light sources is required, which is why the method and the device are expensive.

The object of the invention therefore is to provide a device for the disinfection of ice machines, ice silos, and chutes for transport of ice, which can be manufactured cost-effectively, which enables reliable disinfection of all surfaces coming into contact with the ice, and with which disinfection can also be carried out during the production of ice, particularly for as long as ice or the liquid to be frozen is in the ice machine or in the ice silo.

### THE INVENTION AND ITS ADVANTAGES

The inventive device and the inventive method are characterized by the fact that a disinfectant is atomized into small particles with an atomizer device and is mixed with a carrier fluid to produce a carrier fluid/disinfectant mixture. The carrier fluid/disinfectant mixture is subsequently introduced into the ice machine, the ice silo, and/or the ice chutes for transport of ice. For this purpose, the ice machine, the ice silo, or the ice chute is provided with an inlet for the carrier fluid/disinfectant mixture. If an atomizing nozzle is used as the atomizer device, this device serves not only for the atomization of the disinfectant but also for mixing the carrier fluid and the atomized disinfectant and introducing the carrier fluid/disinfectant mixture into the ice machine, the ice silo, or the ice chute.

If air is used as the carrier fluid, an aerosol consisting of air and disinfectant particles is created. An aerosol is a dynamic system and is subject to continuous changes because of the condensation of vapors on any already existing particles, evaporation of liquid components of the particles, coagulation of small particulates into large ones, and the deposition of particulates on surrounding objects. The carrier fluid/disinfectant mixture behaves like a gas and diffuses throughout the entire ice machine, the entire ice silo, or the entire chute for transport of ice. For this purpose, one or only a few atomizing nozzles are sufficient. Furthermore, the particles of the mixture become attached to the pathogens. In this process, because of the interaction with the disinfectant, the pathogens are rendered harmless.

The characteristic of aerosols that is comparable to that of gases is conditional upon the small diameter. When the diameter is reduced, the space and the mass are decreased by the power of three. The cross-sectional area decreases only by the power of two, however. The settling rate of the particles depends on their gravitational force and the air resistance. Whereas the gravitational force is determined by the mass of the particles, the air friction depends on the cross-sectional area surface and the velocity. Halving the particle diameter reduces the settling rate by a factor of 0.7. The result is that the smaller the diameter of the particles, the better the distribution of the carrier fluid/disinfectant mixture in the space to be disinfected. A diameter of 200 μm constitutes a critical limit in this regard. Below this diameter, the particles distribute themselves like a gas in the space to be disinfected. In contrast to the cleaning devices from prior art, the parts of the ice machine or the ice silo to be disinfected no longer have to be introduced into the spray cone of the atomizing nozzle. Since the carrier fluid/disinfectant mixture becomes distributed in the respective space similar to a gas, one inlet or a few inlets are sufficient to fill the entire space with the mixture and to disinfect the components located within the space.

The disinfectant must comply with the requirement that it kill pathogens. If in addition to that the persistent forms of the pathogens, such as spores are also removed, then sterilization will result. When selecting the disinfectant, the area in which the ice made by the ice machine will be used must also be considered. Since the disinfection also takes place during the operation of the ice machine and the collection of the ice in the ice silo, the disinfectant must not have a lasting effect on the quality of the ice.

In order to enhance the introduction of disinfectant particles that are of the smallest possible diameter into the carrier fluid, the carrier fluid is passed under pressure along a feed inlet of the disinfectant. In the process, small particles of the disinfectant are carried along and are transported in the carrier fluid in the direction of flow. The pressure is above the atmospheric pressure and is typically in the order of 0.5 to 5.0 bar. For pressurization, a compressor, a gas bottle, a pump, or an existing compressed air system on site is used, for example.

In principle, gases and liquids are suitable carrier fluids. The use of air as a carrier fluid is particularly cost-effective. In addition, other gases or steam can be used as carrier fluid. Steam has the advantage that it heats up the disinfectant and because of its higher temperature results in the disinfectant killing the pathogens at a higher level of efficiency. Compared to air, however, supplying pressurized steam involves a somewhat greater expense.

According to an advantageous embodiment of the invention, the atomizer device is an atomizing nozzle. This has a connection for the pressure line, an inlet for the disinfectant, and a nozzle orifice for expanding the carrier fluid/disinfectant mixture into the ice machine, the ice silo, or the ice chute. Advantageously, this pressure line leads into a flow duct. The nozzle orifice is arranged on the end of the flow duct facing away from the pressure line. The disinfectant storage container is connected to the flow duct by means of a feed-line or a metering device. During the flow of the carrier fluid within the flow duct, the carrier fluid carries along particles of the disinfectant. As a function of the pressure of the carrier fluid and the quantity of the disinfectant supplied, a mixing ratio of carrier fluid and disinfectant is established. Consequently, additional mixing or metering is not necessary.

According to a further advantageous embodiment of the invention, the flow duct is provided with an obstruction designed to create turbulence. The obstruction can, for example, protrude into the flow duct transverse to the direction of flow. The laminar flow of the carrier fluid is thereby changed into a turbulent flow. This promotes the mixing process and atomization of the disinfectant in the carrier fluid.

According to a further advantageous embodiment of the invention, the atomizer device is a contact atomizer, a trickling atomizer, a disk atomizer, a steam atomizer, an ultrasonic atomizer, or a Klingenburg atomizer. With a contact or trickling atomizer, the disinfectant is trickled across a porous surface. The carrier fluid flows past the porous surface and picks up small particles of the disinfectant. This process is enhanced by the evaporation of the disinfectant on the porous surface. With a disk atomizer, the disinfectant is applied to a rotating disk. Due to the centrifugal force, small particles of the disinfectant are centrifuged outward as fine mist from the disk and are picked up by the carrier fluid flow. With a steam atomizer, liquid disinfectant is heated such that it changes into the gaseous state. For this purpose, electrode systems are also used; they utilize the conductivity of the disinfectant in order to heat it. The gaseous disinfectant is introduced into the flowing carrier fluid. With an ultrasonic atomizer, a membrane or plate is brought to high frequency oscillation. These vibrations are transferred to the liquid disinfectant. In this process, small particles are knocked out of the liquid disinfectant which are then picked up by the flowing carrier fluid. With a Klingenburg atomizer, vortex guide vanes generate stable horizontal vortices. A liquid disinfectant is injected under high pressure into the centers of the vortices. The vortices atomize the disinfectant into minute particles. The flowing carrier fluid carries along the small particles.

According to a further advantageous embodiment of the invention, the atomizer device is provided with a device for electrostatically charging the atomized disinfectant. In addition, the surfaces of the ice machine, the ice silo, or the ice chutes that are to be disinfected are charged electrostatically, so that the surfaces electrostatically attract the particles of the disinfectant. In this context it should be noted that the particles are positively charged and the surfaces are negatively charged, or vice versa.

According to a further advantageous embodiment of the invention, the storage container is provided with hydrogen peroxide as disinfectant. Hydrogen peroxide has the advantage that it can be mixed with water at any ratio and that it decomposes into water and oxygen at room temperature. It is a strong oxidizer, a weak acid, and is highly toxic for many microorganisms. At a concentration of less than 8%, hydrogen peroxide is harmless for any persons who come into contact with the disinfectant. The disinfectant effect occurs during the decomposition of hydrogen peroxide into water and oxygen. This effect is based upon the reactive atomic oxygen generated during the decomposition. This results in oxidative damage of cellular constituents of the microorganisms, in particular through oxidative cross-linking of proline-rich cell wall proteins and through inactivation of catalytic cysteine residuals in active centers of enzymes. This results in the killing of the cells in the microorganisms. Pathogens are therefore reliably rendered harmless. In the process, the fact that the particles of the finely atomized hydrogen peroxide attach to the pathogens is utilized, whereby an interaction between the pathogens and the hydrogen peroxide is guaranteed, resulting in the destruction of the pathogens.

Hydrogen peroxide compared to other disinfectants stands out in that it decomposes into water and oxygen and can therefore be used without any concern and without limitation for ice machines and ice silos used in the manufacture and processing of foodstuffs. The disinfection process can therefore be carried out even during ice production and at times when ice and liquids to be frozen are present in the ice machine or the ice silo. An additional rinsing step after the disinfection, for removing the disinfectant from the ice machine and the ice silo, is not required. The decomposition products, oxygen and hydrogen, which remain in the ice machine or the ice silo following the disinfection process, do not affect the quality of the ice. The ice meets the higher requirements which are applicable in the food industry.

Apart from the hydrogen peroxide, other disinfectants can also be used either alternatively or cumulatively. A solution using a low concentration of silver particles is also suitable. For applications in the foodstuffs industry field, the concentration must be low enough so that it does not negatively affect the quality of the ice. Furthermore, alcohols can be used as disinfectants.

According to a further advantageous embodiment of the invention, the storage container for the disinfectant is connected with the atomizing nozzle through a disinfectant feed-line. The storage container can, for example be a canister, from which the disinfectant is withdrawn with a suction lance, for example. The feed-line, is a traditional line for liquids, such as a hose or a pipe which connects the suction lance with the atomizing nozzle. If in this process the storage container is located on the same level as the atomizing nozzle, then the disinfectant simply runs by gravity from the storage container to the nozzle on its own without any external auxiliaries. Through changing the level of the storage container relative to that of the atomizing nozzle, the velocity with which the disinfectant is transported from the storage container to the atomizing nozzle can be varied. This in turn has an effect on the quantity of disinfectant in the carrier fluid/disinfectant mixture which is produced in the atomizing nozzle.

According to a further advantageous embodiment of the invention, a heating device is arranged on the atomizing nozzle or on the disinfectant feed-line. This can be a flow-through heater, for example. The nozzle of the disinfectant feed-line can also be provided with a heating wire or a heating sleeve. In order to prevent the disinfectants from cooling again before atomization, care should be taken that the heating device is arranged as closely as possible to the atomizer nozzle. If the disinfectant is heated to a temperature between 40 and 55° C., a significantly higher disinfectant efficiency for killing pathogens can be achieved.

According to a further advantageous embodiment of the invention, a heating device is arranged on the pressure line for the carrier fluid feed. This can be provided as an alternative or an addition to the heating device on the disinfectant feed-line or the atomizer nozzle. This, too, will result in heating-up of the disinfectant and thus an increase in the disinfection process efficiency.

According to a further advantageous embodiment of the invention, the device is provided with a metering device for metering the disinfectant into the carrier fluid. Such metering device can be provided with a metering element, for example, which withdraws a pre-defined quantity of disinfectant from the storage container and introduces it into the pressurized carrier fluid flow. For this purpose, the metering element can have a cavity, for example, which is filled in the storage tank with liquid disinfectant. In a preferred manner, the cavity has an inlet and outlet. The metering element in the pressurized carrier fluid flow is oriented in such a way that the inlet and the outlet are aligned in the direction of flow. The flowing carrier fluid pulls the disinfectant in the cavity out of the cavity.

According to a further advantageous embodiment of the invention, the device is provided with a mixing device for mixing the disinfectant with the carrier fluid. In this instance, the device can be a special mixing chamber, for example, which is additionally provided with devices which produce turbulence of the carrier fluid and therefore a mixing of carrier fluid and disinfectant.

An ice machine in accordance with the invention or an ice silo in accordance with the invention is provided with a device in accordance with the invention for disinfection such that at least one atomizer device is integrated into the housing. The inlet for the carrier fluid/disinfectant mixture is advantageously located in a housing element which borders the interior of the ice machine, the ice silo, and/or the ice chutes. In this way, the finely atomized carrier fluid/disinfect and mixture can be fed into the interior of the ice machine, the ice silo, or the ice chute. The remaining parts of the device for disinfecting are located outside of the space to be cleaned. They should, however, advantageously be integrated into housing bordering the ice machine, the ice silo or the ice chute.

Additional advantages and advantageous embodiments of the invention can be found in the following specification, the drawing, and the claims.

### DRAWING

The drawing represents an embodiment of a device for disinfection of an ice chute and an ice silo. The drawing further shows different embodiments of the ice machine with an ice shaft and ice silo which are provided with a disinfection device in accordance with the invention, as follows:

FIG. 1: Structure of a device for disinfecting an ice shaft and an ice silo,

FIG. 2: First embodiment of an ice machine with ice shaft and ice silo, depicted perspectively,

FIG. 3: Ice machine with ice shaft and ice silo as in FIG. 2 as a side view,

FIG. 4: Ice machine with ice shaft ice silo as in FIG. 2 as a front view,

FIG. 5: Ice machine with ice shaft and ice silo as in FIG. 2 as a plan view,

FIG. 6: Second embodiment of an ice machine with ice shaft and ice silo as a side view,

FIG. 7: Third embodiment of an ice machine with ice shaft as a front view

FIG. 8: Fourth embodiment of an ice machine with ice shaft and ice silo as a front view,

FIG. 9: Ice machine with ice shaft and ice silo as in FIG. 8 as a side view.

### DESCRIPTION OF THE EMBODIMENTS

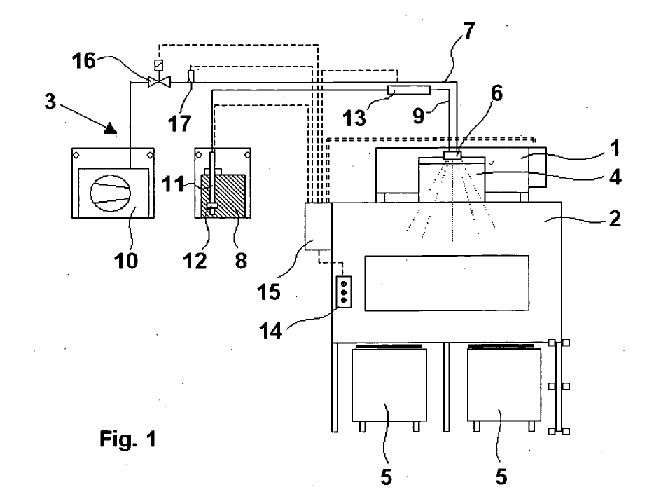

FIG. 1 represents the basic structure of an ice machine 1, an ice silo 2, and a device 3 for disinfecting the ice machine 1 and the ice silo 2. The ice machine 1 is a flake ice machine in which a rotating freezing cylinder is immersed in a tank which is partially filled with water. The ice layer forming on the surface of the freezing cylinder is continuously separated with the help of a scraper. The freezing cylinder, the tank, and the scraper are located in the housing of the ice machine 1 and are not visible in the drawing. The flake ice separated from the freezing cylinder surface drops through an ice shaft 4 serving as an ice chute into the ice silo 2 from where it is moved to the two ice transport carts 5. By means of these ice silos, the ice made by ice machine 1 is collected and moved for further processing or to the point of use. The device 3 for disinfecting essentially consists of an atomizer nozzle 6, a pressure line 7 for supplying compressed air, a storage container 8 with hydrogen peroxide, and a feed-line 9, which connects the storage container 8 with the atomizer nozzle 6. The pressure line 7 also leads into the atomizer nozzle 6. The compressed air is generated with an air compressor 10. The hydrogen peroxide serving as a disinfectant is withdrawn from the storage container 8 by means of a suction lance 11 and is provided to feed-line 9. Since the atomizer nozzle 6 and the storage container 8 are at least approximately on the same level, the hydrogen peroxide is conveyed to the atomizer nozzle without further auxiliaries. A float switch 12 determines whether sufficient hydrogen peroxide is available in the storage container. When the level drops below a pre-defined limit value, this deficiency is indicated to users. On the feed-line 9 which connects the storage container 8 with the atomizer nozzle 6, a flow-through heater 13 is arranged as a heating device. It heats up the hydrogen peroxide supplied to the atomizer nozzle so that it can be introduced into the ice shaft 4 at a temperature of approximately 40 to 50° C.

The atomizer nozzle is arranged in the upper area of the ice shaft 4, and is facing downward. The finely atomized hydrogen peroxide mixed with the compressed air in the atomizer nozzle 6 is introduced into the ice shaft 4, where—because of its physical characteristics which are comparable to those of a gas—it becomes distributed throughout the entire space of the ice shaft 4 and the ice silo 2.

An operating control 14 for the device for disinfection is arranged on the housing of the ice silo 2. On this control station, the user can either manually specify the start and the end of a disinfection procedure or initiate an automatic disinfection at selected periodic intervals. The operating control 14 is connected to a control system 15. This actuates a solenoid valve 16 and a pressure switch 17 on the pressure line 7 when the disinfection process is started. In addition, the suction lance 11 is activated in order to draw-in hydrogen peroxide from the storage container 8. If so desired, the user can switch on the flow-through heater 13 for heating-up the hydrogen peroxide. In addition, the control system 15 is connected to the ice machine 1. The connection of the control system 15 to the different components of the device 3 for disinfection and to the ice machine is indicated by dotted lines in FIG. 1.

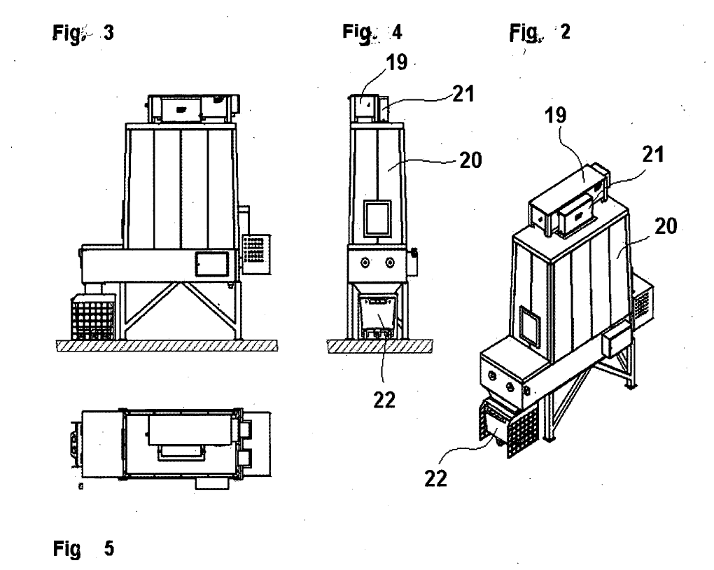

FIGS. 2 to 9 illustrate different embodiments of the ice machines, ice silos, and ice shafts which are provided with a device 3 for disinfection as in FIG. 1. On the first embodiment as in FIGS. 2 to 5, an ice machine 19 is located on top of an ice silo 20. The ice produced by the ice machines 19 is supplied to the ice silo via an ice shaft 21. The ice is transferred into an ice transport cart 22 via the ice silo 20. The device for disinfection is integrated into the housing of the ice silo 20. The device is not visible in FIGS. 2 to 5. The atomizer nozzles are installed in the ice shaft 21, the ice silo 20, and the ice machine 19 such that the disinfectant ejected from the atomizer nozzle can distribute itself in the interior of these units.

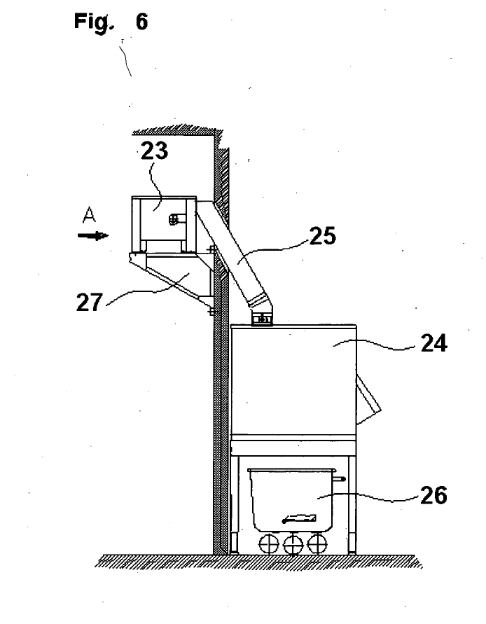

FIG. 6 illustrates a second embodiment of an ice machine 23 with an ice silo 24, an ice shaft 25, and an ice transport cart 26. In contrast to the first embodiment as in FIGS. 2 to 5, the ice machine 23 is not arranged on top of the ice silo 24 but instead on a wall bracket 27. Even though the elements of a device for disinfection are not visible in FIG. 6, they are nevertheless integrated into the constituents of the ice silo 24 and the ice shaft 25, as in the structure shown in FIG. 1.

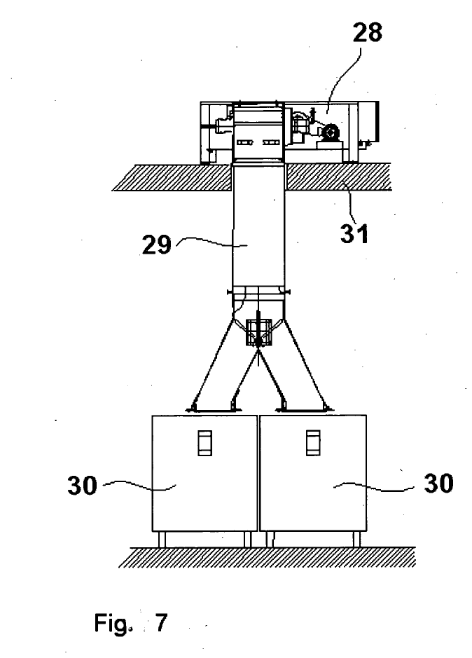

FIG. 7 illustrates a third embodiment of an ice machine 28 with an ice shaft 29 and two ice transport carts 30. In this embodiment, the ice machine 28 is arranged on an intermediate ceiling 31. The ice shaft 29, is a so-called Y-shaft containing a flap valve control with a light barrier. This ensures that the ice manufactured with the ice machine 28 is distributed to the two ice transport carts.

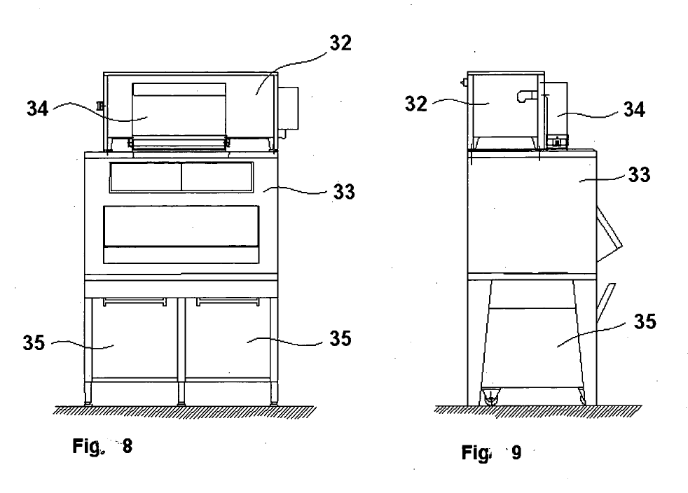

FIGS. 8 and 9 illustrate a fourth embodiment with an ice machine 32, an ice silo 33, an ice shaft 34, and two ice transport carts 35. The ice silo 33 is provided with a door 36, which can be opened for inspection and maintenance purposes.

All features of the invention, either individually or in any given combination, can be essential to the invention.

1. Device for disinfection of ice machines, ice silos, and/or chutes for ice transport
with a storage container for holding a disinfectant,
with a pressure line for the supply of a pressurized carrier fluid,
with at least one atomizer device for atomization of the disinfectant and for generating a mixture of carrier fluid and disinfectant, and
with an inlet for introducing the carrier fluid/disinfectant mixture into the ice machine, the ice silo or the ice chutes.

2. Device as in claim 1, wherein the atomizer device is an atomizer nozzle.

3. Device as in claim 1, wherein the atomizer device is a contact atomizer, a trickling atomizer, a disk atomizer, a steam atomizer an ultrasonic atomizer, or a Klingenburg atomizer.

4. Device as in claim 1, wherein the carrier fluid particles atomized with the atomizer device have a mean diameter of less than 200 μm, in particular preferably of less than 100 μm.

5. Device as in claim 1, wherein the storage tank is provided with hydrogen peroxide as disinfectant.

6. Device as in one of the previous claims, wherein the storage tank is connected with the atomizer device via a disinfectant feed-line.

7. Device as in claim 1, wherein a heating device is arranged on the atomizer device and/or on the disinfectant feed-line.

8. Device as in claim 1, wherein a heating device is arranged on the pressure line.

9. Device as in claim 1, wherein a compressor for the supply of the carrier fluid is arranged on the pressure line.

10. Device as in claim 1, wherein the carrier fluid is air.

11. Device as in claim 1, wherein it is provided with a metering device for metering of disinfectant into the carrier fluid.

12. Device as in claim 1, wherein it is provided with a mixing device for mixing the disinfectant within the carrier fluid.

13. Device as in claim 2, wherein the atomizer nozzle has a flow duct into which the pressure line leads, in that the nozzle orifice is arranged at the end of the flow duct facing away from the pressure line, and that the flow duct is connected by means of a feed-line or a metering device with the storage tank of the disinfectant.

14. Device as in claim 13, wherein the flow duct is provided with an obstruction for creating turbulence.

15. Method for disinfection of ice machines, ice silos, and/or ice chutes, in particular by use of a device as in one of claim 1, characterized by the following process steps:
in that a carrier fluid is supplied under a pressure which is above the atmospheric pressure,
in that a disinfectant is mixed with the carrier fluid
in that the carrier fluid/disinfectant mixture is finely atomized by means of an atomizer device, where the diameter of the particles is less than 100 μm,
in that the atomized carrier fluid/disinfectant mixture is expanded into the ice machine, the ice silo, and/or the ice chutes for transport of ice.

16. Method as in claim 15, wherein hydrogen peroxide is used as disinfectant.

17. Method as in claim 15, wherein air is used as carrier fluid.

18. Method as in one of claims 15, wherein the disinfectant, the carrier fluid, or the carrier fluid/disinfectant mixture is heated to a temperature above the ambient temperature, prior to expansion.

19. Ice machine for making ice in small pieces, that wherein it is provided with a device as in one of claim 1.

20. Ice silo for collection and storage of ice in small pieces, wherein it is provided with a device as in one of claim 1.
